SEQUENCE LISTING (8 sequences)

SEQ ID NO: 1 - DNA, 20 bp, synthetic construct
  source 1..20: mol_type = other DNA
taggtggtttgtcgcgttgt

SEQ ID NO: 2 - DNA, 20 bp, synthetic construct
  source 1..20: mol_type = other DNA
taggcggcttgtcgcgttgt

SEQ ID NO: 3 - DNA, 20 bp, synthetic construct
  source 1..20: mol_type = other DNA
gatatctgcgcattccaccg

SEQ ID NO: 4 - DNA, 28 bp, synthetic construct
  source 1..28: mol_type = other DNA
tgccacagcttaactgtgggcgtgcggg

SEQ ID NO: 5 - DNA, 28 bp, synthetic construct
  source 1..28: mol_type = other DNA
tctcacggcttaactgtgagcgtgcggg

SEQ ID NO: 6 - DNA, 28 bp, synthetic construct
  source 1..28: mol_type = other DNA
actcacagcttaactgtgggcgtgcggg

SEQ ID NO: 7 - DNA, 29 bp, synthetic construct
  source 1..29: mol_type = other DNA
catgcgagactgacctgagctaggagctg

SEQ ID NO: 8 - DNA, 92 bp, synthetic construct
  source 1..92: mol_type = other DNA
taggcggcttgtcgcgttgtctatgcatgcgagactgacctgagctaggagctgaacttgcctaggtgctagcggtggaatgcgcagatatc

(19) United States
(12) Patent Application Publication
(10) Pub. No.: US 2026/0078432 A1
(43) Pub. Date: Mar. 19, 2026

(54) PRIMER, PROBE, AND KIT FOR BROAD-SPECTRUM DETECTION OF MYCOBACTERIA AND USE THEREOF

(71) Applicant: Wuhan Canvest Biotechnology Co., Ltd., Wuhan (CN)

(72) Inventors: Mingzhen Wang, Wuhan (CN); Meilin Chen, Wuhan (CN); Xiang Luo, Wuhan (CN); Yao Hao, Wuhan (CN); Xiaoying Xing, Wuhan (CN); Yujie Liu, Wuhan (CN); Bing Yuan, Wuhan (CN); Guodong Xu, Wuhan (CN)

(21) Appl. No.: 19/373,769

(22) Filed: Oct. 30, 2025

Related U.S. Application Data
(63) Continuation of application No. PCT/CN2024/089755, filed on Apr. 25, 2024.

(30) Foreign Application Priority Data
Sep. 14, 2023 (CN) 2023111866011

(51) Int. Cl. C12Q 1/6825; C12Q 1/6806; C12Q 1/6848; C12Q 1/6851; C12Q 1/689

(52) U.S. Cl. CPC C12Q 1/6825; C12Q 1/6806; C12Q 1/6848; C12Q 1/6851; C12Q 1/689

(57) ABSTRACT

The present disclose provides a primer, probe, and kit for broad-spectrum detection of mycobacteria and use thereof, relating to the technical field of broad-spectrum detection of mycobacteria; the primer-probe combination including forward primer, reverse primer and probe, the nucleotide sequence of forward primer is shown in SEQ ID NOs. 1 and 2, the nucleotide sequence of reverse primer is shown in SEQ ID NO. 3, and the nucleotide sequence of detection probe is shown in SEQ ID NOs. 4-6. The primer-probe combination provided by the present disclose can detect more than 160 types of mycobacteria, covering a wide range; the detection time is greatly shortened; having strong specificity, and has no cross reaction with non-mycobacterial cells and viral genomes; having high detection sensitivity, with the detection limit reaching 100 CFU/ml and the genome detection limit being 10-20 copies/reaction; having high durability and high resistance to matrix interference.

### FIELD OF THE DISCLOSURE

The present disclosure relates to the technical field of broad-spectrum detection of mycobacteria, especially relating to a primer, probe, and kit for broad-spectrum detection of mycobacteria and use thereof.

### STATEMENT REGARDING SEQUENCE LISTING

The sequence listing associated with this application is provided in text format in lieu of a paper copy and is hereby incorporated by reference into the specification. The name of the XML file containing the sequence listing is ZL254612-USP1.xml. The XML file is 8,223 bytes; is created on Oct. 24, 2025; and is being submitted electronically via patent center.

### BACKGROUND

Mycobacterium are a class of bacillus that have no flagella, no spore, slender and slightly curved, it grows in a branched manner. Mycobacteria are divided into three categories: Mycobacterium tuberculosis complex (MTC), Mycobacterium lepare and non-tuberculous mycobacteria (NTM). According to the publication of LPSN (https://lpsn.dsmz.de/), there are currently 266 species of mycobacteria with 24 subspecies. Diseases caused by mycobacterial infection are chronic and accompanied by granulomas, such as tuberculosis and leprosy, which pose a major threat to human health. Non-tuberculous mycobacteria widespread in environmental water and soil are conditionally pathogenic bacteria, which can cause human lung infections, lesions of tissues and organs such as lymph nodes, bones, joints, skin and soft tissues, and even systemic disseminated diseases. In recent years, the prevalence of infectious diseases caused by non-tuberculous mycobacteria has increased worldwide. An increasing number of non-tuberculous mycobacteria have been reported to be pathogenic and potentially risky. About 50 species of pathogenic mycobacteria have been reported to be isolated from the clinic and the environment.

Regulatory agencies such as the State Food and Drug Administration require cell banks, cell therapy biological products and other products to be tested for mycobacterial contamination. The conventional detection method for mycobacteria is culture or guinea pig. The culture method is to inoculate the sample to be tested in solid medium at 37° C. constant temperature for 56 days, and the guinea pig method requires guinea pigs to be inoculated for 42 days before autopsy. Both the culture method and the guinea pig method have problems such as long detection cycle, slow growth, low separation rate and easy contamination, which cannot meet the needs of rapid inspection and release of stem cell therapeutic biological products with high timeliness requirements and high sample cost. FDA guidelines, the European Pharmacopoeia, and the Chinese Pharmacopoeia clearly state that mycobacteria can be detected by a fully validated nucleic acid amplification (NAT) method. Therefore, based on the technical idea of nucleic acid amplification (NAT), there is an urgent need for the development of mycobacteria detection technology in our country to provide a simple, efficient, reproducible and sensitive broad-spectrum detection method specifically for the vast majority of mycobacteria.

### SUMMARY

The qPCR method in the NAT method has the advantages of simplicity, high efficiency, good reproducibility and high sensitivity, and is widely used in the quality control detection of biological products at home and abroad. The present disclosure establishes a TaqMan fluorescence quantitative PCR primer-probe combinations, kits, and detection methods for rapid broad-spectrum detection of over 160 types of mycobacteria, and mycobacterial contamination detection can be completed within a few hours, greatly meeting the time-sensitive detection needs of biological products such as cell banks, virus seed banks, and cell therapy products. Specifically, it is achieved through the following technologies.

The primer-probe combination for broad-spectrum detection of mycobacteria, including forward primer, reverse primer and probe, the nucleotide sequence of the forward primer is shown in SEQ ID NO. 1 and SEQ ID NO. 2, the nucleotide sequence of the reverse primer is shown in SEQ ID NO. 3, the nucleotide sequence of the detection probe is shown in SEQ ID NO. 4, SEQ ID NO. 5 and SEQ ID NO. 6; the 5′ end of the detection probe is coupled with a first fluorescence group, and the 3′ end is coupled with a quenching group.

Preferably, the first fluorescence group is FAM, TET, NED, ROX, CY3, CY5, VIC, JOE, or HEX fluorescence group; the quenching group is TAMRA, NFQ, ECLIPSE, DABCYL, BHQ1, or BHQ2 quenching group.

Preferably, the first fluorescence group is FAM fluorescence group, and the quenching group is BHQ1 quenching group.

More preferably, preparing products for broad-spectrum detection of mycobacteria.

A kit for broad-spectrum detection of mycobacteria, including any of the primer-probe combination described above.

Preferably, the above kit also includes an internal control probe and an internal control plasmid, the nucleotide sequence of the internal control probe is shown in SEQ ID NO. 7, and the nucleotide sequence of the target fragment in the internal control plasmid is shown in SEQ ID NO. 8; the 5′ end of the internal control probe is coupled with a second fluorescence group, and the 3′ end is coupled with a quenching group, and the type of the second fluorescence group is different from the first fluorescence group.

A method for broad-spectrum detection of mycobacteria that is not intended for disease diagnosis and treatment, using any of the above kit to detect.

Preferably, the above method for broad-spectrum detection of mycobacteria includes the following steps:
Choosing whether to perform a pretreatment process according to the type of sample to be tested;
If there is no need for sample pretreatment, it is directly prepared as a test solution for nucleic acid extraction;
If sample pretreatment is required, the pretreatment process is to add cell lysate treatment (optionally, lysis for about 30 minutes), and collect the pellet as a test product after centrifugation;
The internal control plasmid is added to the test product or qPCR reaction solution, and the nucleic acid in the test product is extracted; the positive control plasmid was added to the internal control plasmid and nuclease-free water and diluted to form a positive control;
Taking sterile normal saline as a negative control (NCS), adding an internal control plasmid (IC) to extract it simultaneously with the test product, or adding an internal control plasmid (IC) to the qPCR reaction after extracting nucleic acid; no template control (NTC) is nuclease-free water, or an internal control plasmid prepared with nuclease-free water;
The first fluorescence group detection channel and the second fluorescence group detection channel were created on the fluorescence quantitative PCR instrument, and qPCR reaction was performed on the test product, no template control (NTC), negative control (NCS), and positive control (PC) respectively, read the Ct value of the two channels respectively;
The result is determined for the Ct value;
The amplification status is checked in the corresponding channel, and the threshold line of the amplification curve of the test product is 10% of the Ct value of the target channel in the positive control, and the threshold line of the amplification curve of the internal control plasmid is 10% of the Ct value of the internal control channel in the no template control or negative control;
The requirements for quality control results are shown in Table 1 below.

TABLE 1
requirements for quality control results

| Quality control | The first fluorescence | The second fluorescence |
|---|---|---|
| samples | group detection channel | group detection channel |
| Positive | Positive (Ct < 35 with | Positive (Ct < 40 with |
| control PC | normal amplification | normal amplification |
| | curve) | curve) |
| Negative | Negative (no Ct value, | Positive (Ct < 40 with |
| control NCS | no normal | normal amplification |
| | amplification curve) | curve) |
| No template | Negative (no Ct value, | Add IC: positive |
| control NTC | no normal | (Ct < 40 with normal |
| | amplification curve) | amplification curve) |
| | | No add IC: negative (no |
| | | Ct value, no normal |
| | | amplification curve) |

The criteria for determining sample test results are shown in Table 2 below.

TABLE 2
Criteria for determining sample test results

| The first fluorescence | The second fluorescence group | |
|---|---|---|
| group detection channel | detection channel | The result is determined |
| Positive (Ct < 40 with normal | Independent | Positive |
| amplification curve) | | |
| Negative (no Ct value, no | Negative (no Ct value, no normal | qPCR inhibition |
| normal amplification curve) | amplification curve) | |
| Boundary value (Ct ≥ 40 with | | |
| normal amplification curve) | | |
| Boundary value (Ct ≥ 40 with | Positive (Ct < 40 with normal | The results are invalid, and |
| normal amplification curve) | amplification curve) | the sample volume can be |
| | | increased assay after |
| | | re-extraction |
| Negative (no Ct value, no | Positive (Ct < 40 with normal | Negative |
| normal amplification curve) | amplification curve) | |

It should be noted that in the positive control of the present disclosure, the type and gene sequence of the positive control plasmid are not limited, and can be determined according to the actual situation. Generally speaking, as long as it is a plasmid containing the target sequence of mycobacteria detection, it can be used as a positive control plasmid of the present disclosure.

Optionally, a mixture of three recombinant plasmids, pUC57—M. tuberculosis, pUC57—M. terrae, and pUC57—M. xenopi, can be selected for the positive control plasmid.

It should be noted that if the internal control plasmid is added during the sample nucleic acid extraction process, the Ct value of the second fluorescence group channel of the test product determined to be negative after testing should be separated from the Ct value of the second fluorescence group channel in the extraction negative control (NCS). +/−3 cycles. If the internal control plasmid is added during the qPCR reaction, the Ct value of the second fluorescence group channel of the test product determined to be negative after testing should be separated from the Ct value of the second fluorescence group channel in the extraction negative control+/−2 cycles. If the second fluorescence group channel signal is inhibited, it needs to be retested or the sample should be treated appropriately to eliminate the inhibitor.

Compared with the prior art, the advantages of the primer-probe combination and the corresponding qPCR detection method provided by the present disclosure are:
1. The primer-probe combination provided by the present disclosure can be used to perform double fluorescence quantitative PCR, and according to the Ct value and amplification curve, it can quickly judge whether there is mycobacterial contamination in the sample, and can detect more than 160 kinds of mycobacteria, covering a wide range;
2. The detection method provided by the present disclosure can complete the detection of mycobacterial contamination within a few hours, and the detection time is greatly shortened compared with the traditional detection method;
3. Strong specificity, no cross-reactivity with human and animal cells, fungi,
other bacteria except mycobacteria, mycoplasma, and common gene therapy-related virus genomes;
4. The detection sensitivity is high, the detection limit reaches 100 CFU/ml, and the detection limit of the genome is 10-20 copies/reaction, and the sensitivity is not lower than the standard of the Chinese Pharmacopoeia.
5. Strong durability and anti-matrix interference ability, 10⁷ 293T/CHO/Vero cells and 1 ml of culture supernatant were introduced, and 100 CFU/ml was extracted mycobacterial genomes can also be detected later.
6. The present disclosure is provided with an internal control system (internal control plasmid and internal control probe), which can monitor whether there is an inhibition phenomenon in the extraction of the sample to be tested and the qPCR reaction when detecting mycobacteria, and can effectively avoid the appearance of false negatives and the result judgment is more accurate and reliable.

### BRIEF DESCRIPTION OF THE DRAWINGS

FIG. 1 shows the detection capability verification results of the mycobacteria qPCR detection system in example 3;

FIGS. 2A-2F show the sensitivity verification results of the standard plasmid in example 3;

FIG. 3 shows the results of cross-reactivity verification between the primer-probe combination of the present disclosure and non-mycobacteria in example 4;

FIGS. 4A-41 show the durability verification results of the mycobacteria detection method in example 5;

FIG. 5 shows the durability verification results of the mycobacteria detection method in example 5;

FIG. 6 shows the specificity verification results of the internal control probe in example 6;

FIG. 7 shows the verification results of whether there is cross-reactivity between mycobacteria detection and internal control plasmid in example 6;

FIG. 8 shows the detection results of the primer-probe combination and detection method of the present disclosure in example 7.

### DETAILED DESCRIPTION OF PREFERRED EMBODIMENTS

TaqMan PCR technology is a type of real-time PCR, compared with traditional PCR, it adds a probe to the reaction system that labels the fluorescent label and quench group at both ends, respectively. When the probe structure is intact, the energy of the fluorescence emitted by the fluorescent label is transferred to the quenching group, showing a quenching effect. If there is a target sequence in the amplification process, the probe molecule is gradually hydrolyzed and cut off, and the fluorescent reporter group and the quenching group dissociate from each other, blocking the fluorescence resonance energy transfer effect between the two, and the fluorescent reporter group emits a fluorescent signal. As amplification progresses, the fluorescence signal enhances linearly as the target fragment is amplified.

The technical solution of the present disclosure will be clearly and completely described below, and it is clear that the described example is only a part of the examples of the present disclosure, not all of the examples. Based on the examples in the present disclosure, all other examples obtained by ordinary technicians in the field without performing creative labor fall within the scope of protection of the present disclosure.

In the primer-probe combination used in the following example, the 5′ end of the detection probe is coupled with a first fluorescence group, and the 3′ end is coupled with a quenching group; the 5′ end of the internal control probe is coupled with a second fluorescence group, and the 3′ end is coupled with a quenching group.

Optionally, the first fluorescence group can be selected from FAM, TET, NED, ROX, CY3, CY5, VIC, JOE, HEX fluorescence group, or any of the other fluorescence group.

Optionally, the second fluorescence group can likewise be selected from FAM, TET, NED, ROX, CY3, CY5, VIC, JOE, HEX fluorescence group, or any other fluorescence group, but the second fluorescence group cannot be the same as the first fluorescence group.

Optionally, the quenching group can be selected from TAMRA, NFQ, ECLIPSE, DABCYL, BHQ1, BHQ2 quenching group, or any of the other quenching group.

The selection of quenching group on the detection probe and the internal control probe is not required, and can be the same or different.

In the following example, the first fluorescence group is selected as the FAM fluorescence group as an example, the second fluorescence group is selected as the HEX fluorescence group as an example, and the quenching group is selected as the BHQ1 fluorescence group as an example.

The detection primers used in the following examples were synthesized by Wuhan Tianyi Huiyuan Biotechnology Co., Ltd. the detection probe, internal control probe and internal control plasmid were synthesized by Bioengineering (Shanghai) Co., Ltd.

RNase-free water used in the following examples, also Nuclease-free water, i.e. no RNA enzymes and no DNases.

The technical schemes adopted in the following examples shall adopt conventional molecular biology techniques unless otherwise specified. Human derived cells (e.g., human embryonic kidney cells 293 and 293T, human umbilical cord mesenchymal stem cells MSC), monkey derived cell lines (e.g., african green monkey kidney cells Vero), hamster derived cell lines (e.g., hamster renal cells BHK-21, hamster ovary cells CHO-K1), bovine derived cell lines (e.g., bovine kidney cells MDBK), Porcine derived cell lines (e.g., porcine kidney cells PK-15) used are stored independently by the company. Adenovirus HAd-5, adeno-associated virus AAV-2, Escherichia coli, Clostridium sporogenes, Staphylococcus aureus, Salmonella typhimurium, Bacillus subtilis, Pseudomonas aeruginosa, Bacillus cereus, Micrococcus luteus, Corynebacterium diphtheria, Mycoplasma pneumoniae, Mycoplasma oral used are stored independently by the company.

Optionally, when the internal control plasmid is added to the test product, the internal control plasmid is 700 copies/sample. When the internal control plasmid is added to the qPCR reaction solution, the internal control plasmid is 200 copies/well.

Optionally, in each reaction well, the positive control plasmid and the internal control plasmid in the positive control are 1000 copies/well and 200 copies/well respectively.

Unless otherwise specified, the reagents used in the following examples (e.g., qPCR reaction solution, reagents, buffers of nucleic acid extraction system, etc.), kits and technical solutions are commercially available reagents, kits, and the completion of routine molecular biology technology operations in the industry.

### Example 1: Synthesis of Primer-Probe

### 1. Design of Mycobacterial Primer-Probe:

Refer to the European Pharmacopoeia, the Chinese Pharmacopoeia, the Guidelines for the Diagnosis and Treatment of Nontuberculous Mycobacteriosis (2020 Edition), related literature and other materials to identify common pathogenic mycobacterial species and download their 16S rRNA nucleic acid sequence from NCBI. The nucleic acid sequences of mycobacterium 16S rRNA were aligned with other bacterial 16S rRNA, mycoplasma 16S rRNA, and fungal 18S rRNA, and primer-probe that could specifically detect mycobacteria were designed, containing two forward primers, one reverse primer and three detection probes, of which the primers are synthesized by Tianyi Huiyuan and the detection probes are synthesized by biotechnology, and the sequences are shown in Table 3 below.

TABLE 3
Nucleotide sequences of primer-probe combination

| Component | Sequence (5′-3′) | |
|---|---|---|
| Forward primer | taggtggtttgtcgcgttgt | As shown in |
| FP1 | | SEQ ID NO. 1 |
| Forward primer | taggcggcttgtcgcgttgt | As shown in |
| FP2 | | SEQ ID NO. 2 |
| Reverse primer | gatatctgcgcattccaccg | As shown in |
| RP | | SEQ ID NO. 3 |
| Detection probe | FAM-tgccacagcttaactg | As shown in |
| probe 1 | tgggcgtgcggg-BHQ1 | SEQ ID NO. 4 |
| Detection probe | FAM-tctcacggcttaactg | As shown in |
| Probe2 | tgagcgtgcggg-BHQ1 | SEQ ID NO. 5 |
| Detection probe | FAM-actcacagcttaactg | As shown in |
| Probe3 | tgggcgtgcggg-BHQ1 | SEQ ID NO. 6 |

### 2. Internal Control Plasmid and Probe Sequence

The target fragment of the internal control plasmid was synthesized and the recombinant plasmid pUC57-IC was constructed, and the plasmid was added to the mycobacterial qPCR detection system as a template, and the nucleotide sequence of the target fragment was shown in SEQ ID NO. 8, and specifically was:

taggcggcttgtcgcgttgtctatgcatgcgagactgacctgagctag
gagctgaacttgcctaggtgctagcggtggaatgcgcagatatc.

The nucleotide sequence of the corresponding internal control probe ProbelC, was shown in SEQ ID NO. 7, and specifically was:

HEX-catgcgagactgacctgagctaggagctg-BHQ1

### Example 2: Establishment of Mycobacterial qPCR Detection System and Addition of Internal Control Plasmid

The pUC57-IC recombinant plasmid was synthesized and the concentration was determined, the copy number of the pUC57-IC plasmid was calculated according to the formula, and the gradient was diluted to 1×10³ copies/μl for later use.

The pUC57—M. xenopi recombinant plasmid shared with the internal control in the qPCR detection system was synthesized, and the concentration was determined, and the copy number of plasmid was calculated according to the formula and gradient diluted to 10 copies/μl. Using TakaRa's Probe qPCR Mix, the qPCR detection reaction system is configured according to Table 4 below to make pUC57—M. xenopi in the reaction well is 100 copies/reaction; the reaction procedure in Table 5 was performed for dual-channel qPCR.

TABLE 4
qPCR detection reaction system

| Component | Usage (μl) |
|---|---|
| Probe qPCR Mix, with UNG (2×) | 15 |
| RP (50 μM) | 0.3 |
| FP1 + FP2 (50 μM) | 0.15 + 0.15 |
| probe1 + probe2 + probe3 + probeIC (20 μM) | 0.3+ 0.3 + 0.3 + 0.3 |
| IC (1000 copies/μl) | 0.2 |
| RNase-free Water + template to be detected | 13 |
| Total volume | 30 |

Note:
If the sample is incorporated with IC during the extraction process, the IC is replaced with RNase-free Water.

TABLE 5
qPCR procedure

| | | |
|---|---|---|
| 1 | cycle | 25° C. for 10 min |
| | | 95° C. for 30 sec |
| 45 | cycles | 95° C. for 5 sec |
| | | 60° C. for 30 sec |

It was confirmed that the addition of IC at 200 copies/reaction did not affect the detection sensitivity Ct value of pUC57—M. xenopi, and the internal control channel can be detected stably.

### Example 3: Detection Capability Verification of Mycobacterial qPCR Detection System

### 1. Prediction of Mycobacteria Detection Range

The designed primer-probe is compared with the Primer-BLAST in NCBI for specific sequence search, and the results show that the primer-probe in the present disclosure can cover 167 types of mycobacteria (including 39 types of pathogenic mycobacteria), and the statistical results are shown in Table 6.

TABLE 6
Statistical results of specific sequence search and comparison

| | NO. of |
|---|---|
| Mycobacterium | Species |
| Mycobacterium tuberculosis complex (MTC) | 5 |
| Mycobacterium leprae | 1 |
| non-tuberculous Mycobacteria (NTM) | 161 |
| Total | 167 |

### 2. Verification of Mycobacteria Detection Range

The 11 types of mycobacteria (M. avium, M. terrae, M. shimoidei, M. kansasii, M. asiaticum, M. scrofulaceum, M. gordonae, M. abscessus, M. fortuitum, M. phlei, M. tuberculosis) in the national reference products in the PCR detection kit of Mycobacterium tuberculosis of the Chinese Academy of Food and Drug Control were used as material, the genomes of 11 types of mycobacterial references were extracted and the qPCR reaction system and qPCR procedure shown in Tables 4 and 5 above was adopted for detection.

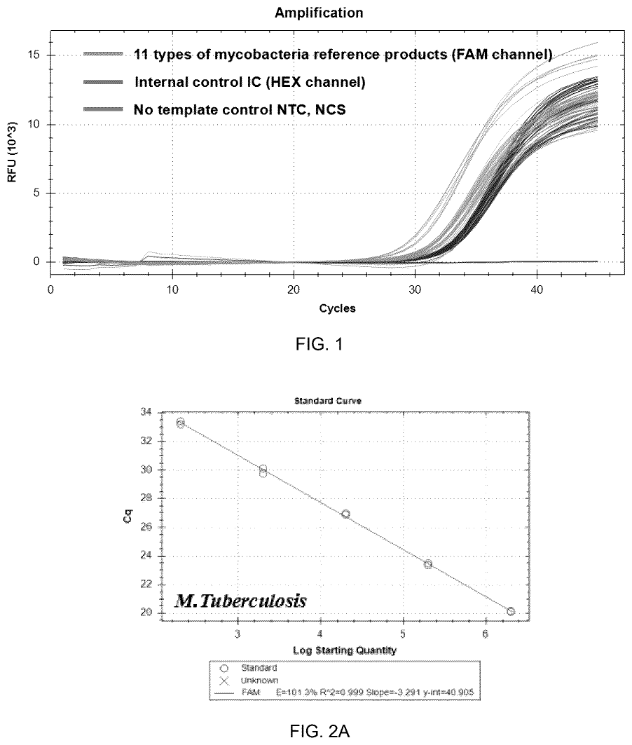

As shown in FIG. 1, 11 types of mycobacteria were detected 100%, and the amplification results are shown in Table 7.

TABLE 7
Summary of amplification

| Mycobacteria | Detection rate (number | Mean |
|---|---|---|
| (1 × 10³ | of detections/total | Ct |
| bacteria/ml) | number of reactions) | value |
| M. avium | 100% (3/3) | 30.61 |
| M. terrae | 100% (3/3) | 31.28 |
| M. shimoidei | 100% (3/3) | 30.63 |
| M. kansasii | 100% (3/3) | 30.47 |
| M. asiaticum | 100% (3/3) | 30.98 |
| M. scrofulaceum | 100% (3/3) | 30.30 |
| M. gordonae | 100% (3/3) | 31.05 |
| M. abscessus | 100% (3/3) | 29.74 |
| M. fortuitum | 100% (3/3) | 30.50 |
| M. phlei | 100% (3/3) | 28.83 |
| M. tuberculosis | 100% (3/3) | 30.59 |

### 3. Verification of the Sensitivity of Standard Plasmids and the Establishment of Standard Curves

The 6 types of mycobacteria (M. tuberculosis, M. lepare, M. scrofulaceum, M. xenopi, M. terrae, M. phlei) were selected for artificially constructed Mycobacterium 16S rRNA standard plasmid, using Plasmid Mini Kit I (OMEGA) to extract Mycobacterium 16S rRNA standard plasmids and measurement of plasmid concentration, calculating plasmid copy number, diluting to 2×10⁶-2×10² copies/μL plasmid standard solution for each point of the standard curve. 2×10¹ copies/μl plasmid standard solution was used as a plasmid sensitivity control.

The qPCR was performed using the amplification system and reaction procedure in Tables 4 and 5 above, on different three days repeating the three times experiment independently, with 8 wells repeated for each experimental plasmid sensitivity control.

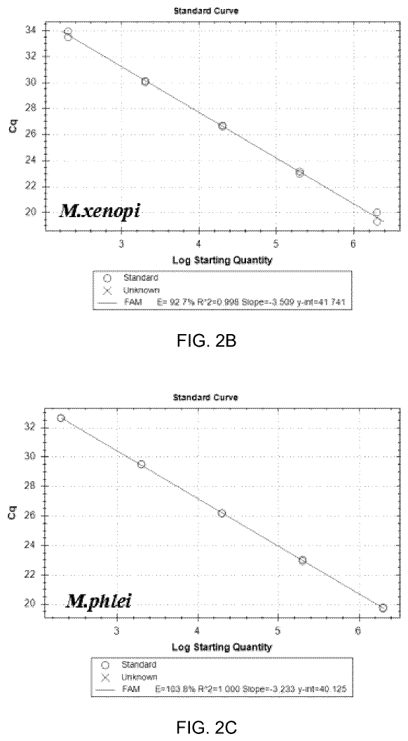

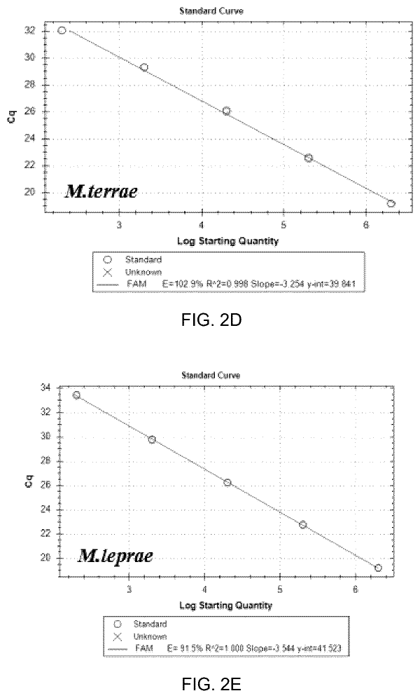

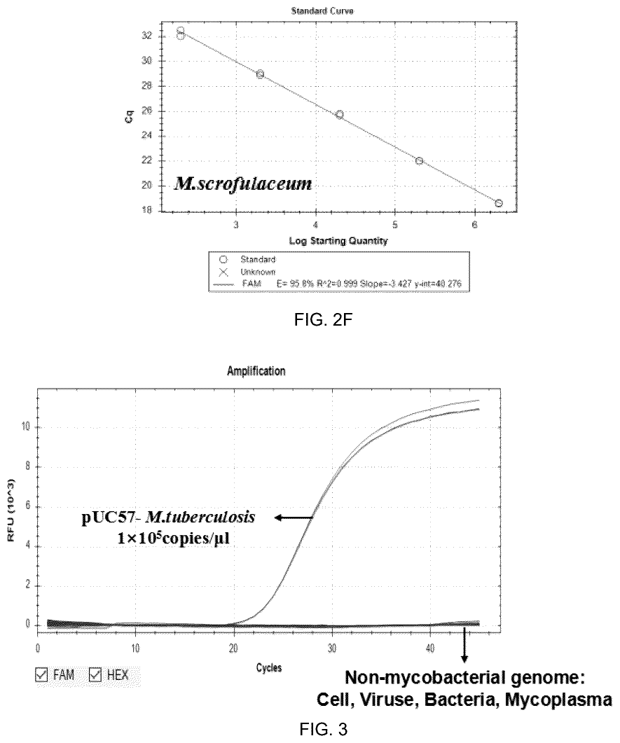

The results showed that the amplification efficiency of the 6 types of mycobacteria was >90%, and the correlation coefficient R²≥0.99, the plasmid sensitivity reached 100 copies/reaction. The experimental results are shown in Table 8 and FIG. 2.

TABLE 8
Verification results of standard plasmid sensitivity

| | | Detection rate (number | Mean |
|---|---|---|---|
| Mycobacteria (100 | fluorescence | of detections/total | Ct |
| copies/reaction) | channel | number of reactions) | value |
| M. tuberculosis | FAM | 100% (24/24) | 34.24 |
| | HEX | | 35.27 |
| M. lepare | FAM | 100% (24/24) | 34.10 |
| | HEX | | 34.92 |
| M. scrofulaceum | FAM | 100% (24/24) | 34.49 |
| | HEX | | 34.95 |
| M. xenopi | FAM | 100% (24/24) | 34.87 |
| | HEX | | 35.19 |
| M. terrae | FAM | 100% (24/24) | 34.08 |
| | HEX | | 35.31 |
| M. phlei | FAM | 100% (24/24) | 34.79 |
| | HEX | | 34.90 |

### 4. Mycobacterial Genome Detection Limit

The 3 types of mycobacteria M. bovis BCG (Shanghai Ruichu Biotechnology Co., Ltd. BCG lyophilized powder), M. phlei (the plate bacteria obtained by our company's culture method) and M. gordonae (purchased by ATCC) for example, using the TaKaRa MiniBEST Bacteria Genomic DNA Extraction Kit Ver. 3.0 (TaKaRa, 9763) to extract genomic DNA, calculating the copy number according to the genome size and measured concentration, and performing gradient dilution; 100, 50, 20, and 10 copies/reaction were used as reaction samples, on different three days repeat the three times experiment independently, and each experiment was repeated for 8 wells at each dilution, and the reaction system and reaction procedure were the same as those in Tables 4 and 5.

The results are shown in Table 9 below, and the detection limit of M. bovis BCG genomic nucleic acid is 20 copies/reaction; the genome detection limit of M. phlei is 10 copies/reaction; the genome detection limit of M. gordonae is 20 copies/reaction.

TABLE 9
Verification results of detection limit

Number of positive responses/total number
of responses (positive rate).

| | 100 | 50 | 20 | 10 |
|---|---|---|---|---|
| Mycobacterial | copies/ | copies/ | copies/ | copies/ |
| genome | reaction | reaction | reaction | reaction |
| M. bovis BCG | 24/24 | 24/24 | 24/24 | 22/24 |
| | (100%) | (100%) | (100%) | (91.67%) |
| M. phlei | 24/24 | 24/24 | 24/24 | 24/24 |
| | (100%) | (100%) | (100%) | (100%) |
| M. gordonae | 24/24 | 24/24 | 24/24 | 20/24 |
| | (100%) | (100%) | (100%) | (83.33%) |

### 5. Verification of the Detection Limit of Mycobacterial Standards

The precision reference material in the national reference product for the PCR detection kit of Mycobacterium tuberculosis of the Chinese Academy of Food and Drug Control (Mycobacterium tuberculosis CMCC93009, 1×10² bacteria/ml), and M. smegmatis standard of M. smegmatis of Beijing Sanyao Technology Development Company (0.5-1×10³ CFU/ml, diluted 10 times before use) as the material, and the mycobacterial genome extraction method developed by our company was used to extract samples 4 times in 4 days 2 replicates each time. After daily extraction, qPCR assay was performed, with 3 double wells per sample and a total of 24 wells in 4 experiments, reaction system and reaction procedure are shown in Tables 4 and 5.

The results are shown in Table 10: the qPCR assay in this protocol can stably detect Mycobacterium tuberculosis (100 bacteria/ml) and mycobacterium smegma (100 CFU/ml), which meets the sensitivity requirements of the current version of the Chinese Pharmacopoeia for mycobacterium detection.

TABLE 10
Verification results of the detection
limit of mycobacterial standards

| | | Detection rate (number | Mean |
|---|---|---|---|
| | fluorescence | of detections/total | Ct |
| Mycobacteria | channel | number of reactions) | value |
| M. tuberculosis | FAM | 100% (24/24) | 32.86 |
| (100 bacteria/ml) | HEX | | 32.98 |
| M. smegmatis | FAM | 100% (24/24) | 30.62 |
| (100 CFU/ml) | HEX | | 32.38 |

### Example 4: Specificity Verification of Mycobacteria Detection Method

### 1. Extraction of Genomic DNA of Various Biological Cells

### (1) Human and Animal Cell Genomic DNA

Porcine kidney cells PK-15, bovine kidney cells MDBK, African green monkey kidney cells Vero, Chinese hamster ovary cells CHO-K1, human embryonic kidney cells 293 and 293T, hamster renal cells BHK-21, and human umbilical cord mesenchymal stem cells MSC, all of which are stored by the company, using QIAampOR Mini kit (50) (QIAGEN, 51304) to extract genomic DNA.

### (2) Viral Genomic DNA

Adenovirus HAd-5 and adeno-associated virus AAV-2 are stored by the company, using the Viral Nucleic Acid purification kit (simgen, 4002050) to extract genomic DNA.

### (3) Bacterial Genomic DNA

Escherichia coli, Clostridium sporogenes, Staphylococcus aureus, Salmonella typhimurium, Bacillus subtilis, Pseudomonas aeruginosa, Bacillus cereus, Micrococcus luteus, Corynebacterium diphtheria, all of which are stored by the company, using TaKaRa MiniBEST Bacteria Genomic DNA Extraction Kit Ver. 3.0 (TaKaRa, 9763) to extract genomic DNA.

### (4) Common Mycoplasma Genomic DNA

Mycoplasma pneumoniae, Mycoplasma oral are stored by the company, using TakaRa MiniBEST Bacteria Genomic DNA Extraction Kit Ver. 3.0 (TaKaRa, 9763) to extract genomic DNA.

### 2. Preparation of Working Concentration Genomic DNA

The concentration of genomic DNA of the above mentioned cells, bacteria, and mycoplasma was measured by microspectrophotometer. According to the concentrations measured, bacterial and mycoplasma genomes were diluted to 0.2 ng/μl with RNase-free Water, human and animal cell genomes were diluted to 20 ng/μl, adenovirus HAd-5, adeno-associated virus AAV-2 was extracted from 1 ml physical titer of 10⁸ copies/ml viral fluid to extract DNA, then take 5 μl as a template to participate in qPCR. The pUC57—M. tuberculosis recombinant plasmid was used as the positive control plasmid.

The qPCR reaction system and amplification procedure are the same as those in Tables 4 and 5 above.

The results are shown in FIG. 3, and it can be seen that the primer-probe combination of the present disclosure has no cross-reactivity with non-mycobacteria; the summary of non-mycobacterial genomic DNA amplification is shown in Table 11.

TABLE 11
Common non-mycobacterial cells

| Human and | | | Common |
|---|---|---|---|
| animal cells | | Other bacteria | mycoplasma |
| (100 ng/reaction) | Virus | (1 ng/reaction) | (1 ng/reaction) |
| PK-15, MDBK, | HAd-5, | Escherichia coli | Mycoplasma |
| Vero, CHO-K1, | AAV-2 | Clostridium | pneumonia |
| 293, 293T, | | sporogenes | Mycoplasma |
| BHK-21, MSC | | Staphylococcus | Oral |
| | | aureus | |
| | | Salmonella | |
| | | typhimurium | |
| | | Bacillus subtilis | |
| | | Pseudomonas | |
| | | aeruginosa | |
| | | Bacillus cereus | |
| | | Micrococcus luteus | |
| | | Corynebacterium | |
| | | diphtheria | |

Determination of Ct value: NA (no cross-reactivity)

### Example 5: Durability Verification of Mycobacterial Detection Method

Mycobacteria are intracellular bacteria, and when detecting cell samples contaminated with mycobacteria, it is necessary to co-extract the culture supernatant and cell lysis for detection, and the cell DNA introduced during the extraction process may inhibit the amplification of mycobacterial targets and affect the detection, so durability verification is required.

293T/CHO/Vero cell genomes were extracted, concentrations were measured and diluted to 80 ng/μl for later use. The plasmid standards of M. terrae, M. xenopi, and M. tuberculosis with 100 copies/reaction were used as controls. 400 ng/reaction of 293T/CHO/Vero cell genomes was introduced in 100 copies/reaction plasmid standards of M. terrae, M. xenopi, and M. tuberculosis, respectively, all the above samples were tested by mycobacterial qPCR, and the qPCR reaction system and reaction procedure are shown in the tables 4 and 5.

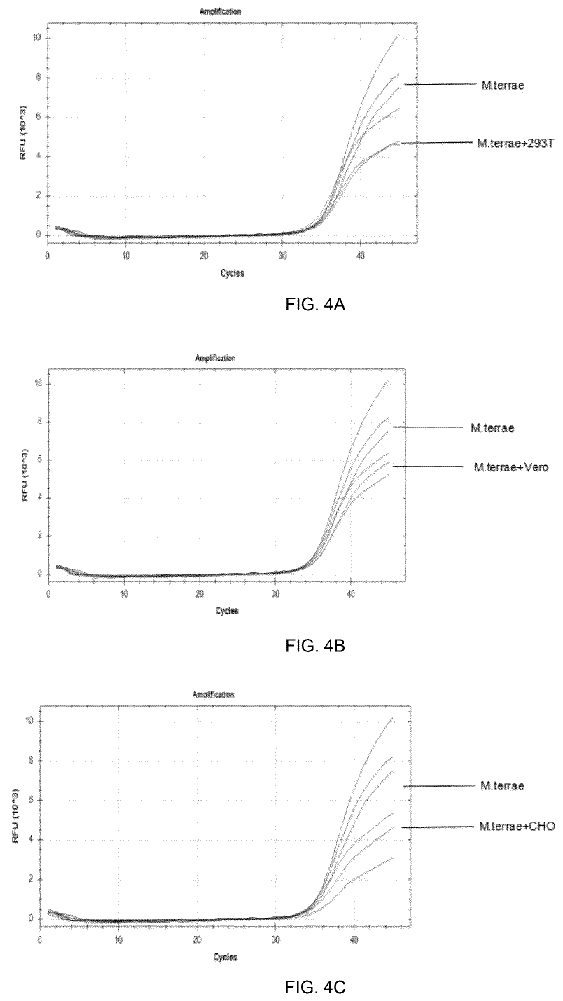

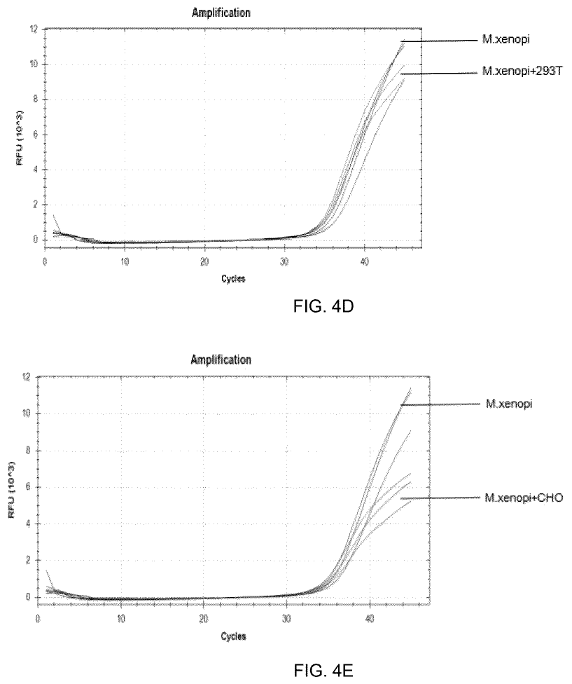

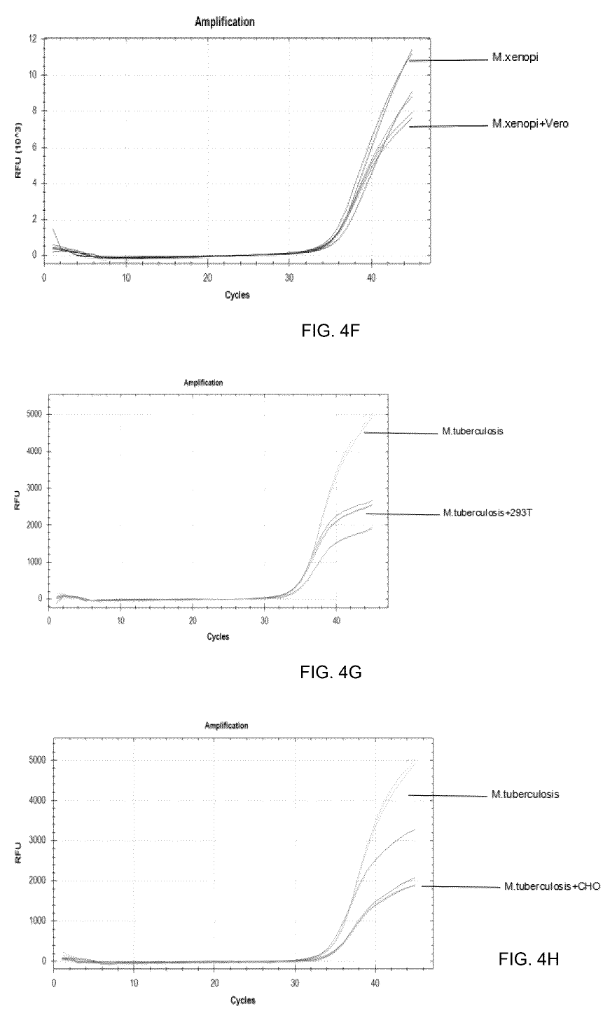

The validation results are shown in FIG. 4 and Table 12, compared to a control without the 293T/CHO/Vero cell genome, the introduction of the 293T/CHO/Vero cell genome will not be resulted in false negatives for mycobacterial tests.

TABLE 12
Durability verification results of mycobacteria detection method

| | Mean Ct value (FAM/HEX) | | | |
|---|---|---|---|---|
| | M. | | | |
| | Tuberculosis | M. Terrae | M. xenopi | |
| | (100 | (100 | (100 | |
| Experimental | copies/ | copies/ | copies/ | Detection |
| grouping | reaction) | reaction) | reaction) | rate |
| Mycobacteria | 33.86/34.22 | 34.19/35.22 | 34.64/35.25 | 100% |
| | | | | (n = 3) |
| Mycobacteria + | 33.20/33.73 | 33.73/35.06 | 33.95/35.14 | 100% |
| 400 ng/reaction | | | | (n = 3) |
| 293T DNA | | | | |
| Mycobacteria + | 33.33/33.66 | 34.02/34.74 | 33.73/34.72 | 100% |
| 400 ng/reaction | | | | (n = 3) |
| CHO DNA | | | | |
| Mycobacteria + | 33.43/34.17 | 34.22/34.75 | 34.50/34.97 | 100% |
| 400 ng/reaction | | | | (n = 3) |
| Vero DNA | | | | |

The precision reference material of M. tuberculosis (100 types of bacteria) in the PCR detection kit of Mycobacterium tuberculosis of the Chinese Academy of Food and Drug Control, and M. smegmatis standard (100 CFU) of Beijing Sanyao Technology Development Company as the material, artificially contaminated with 1 ml of culture supernatant 10⁷ samples of 293T/CHO/Vero cells. Mycobacterial genome extraction method developed by the company was used to extract mycobacterial genome nucleic acid for mycobacterial qPCR detection, and the qPCR reaction system and reaction procedure are the same as those in Tables 4 and 5.

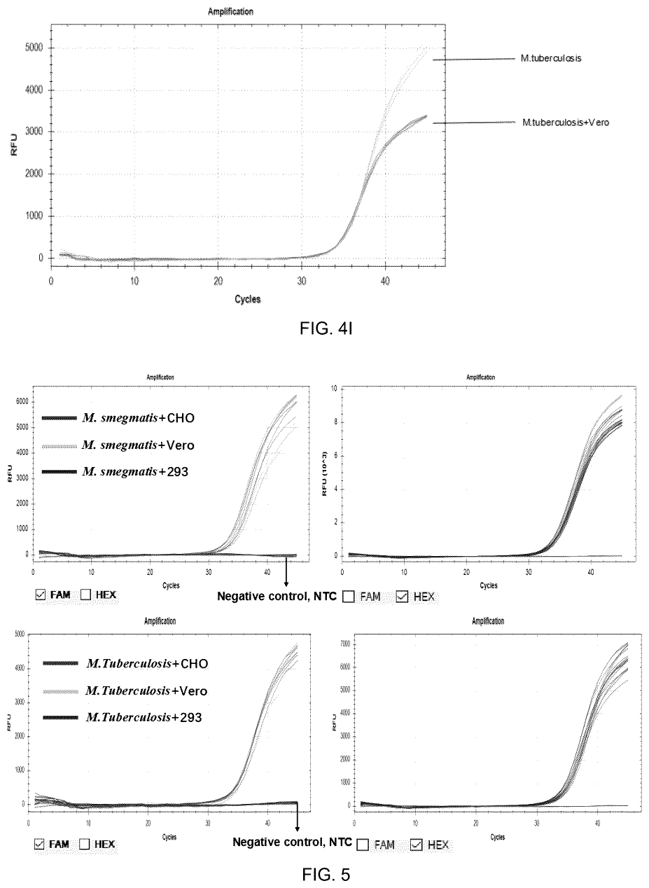

The verification results are shown in FIG. 5 and Table 13, the cell samples without mycobacterium did not show amplification signals, and the samples of artificially contaminated M. tuberculosis and M. smegmatis were positive.

TABLE 13
Durability verification results of mycobacteria detection method

| | | Mean Ct | | |
|---|---|---|---|---|
| | | value | Mean Ct | Detection |
| Experimental grouping | | (FAM) | value (HEX) | rate |
| 10⁷ 293T/CHO/Vero | | NA | 33.45/33.80/33.81 | 0 |
| M. | +1 × 10⁷ | 33.70 | 33.25 | 100% |
| tuberculosis | 293T | | | (n = 3) |
| | +1 × 10⁷ | 33.83 | 33.38 | 100% |
| | CHO | | | (n = 3) |
| | +1 × 10⁷ | 33.52 | 33.53 | 100% |
| | Vero | | | (n = 3) |
| M. | +1 × 10⁷ | 33.11 | 32.92 | 100% |
| smegmatis | 293T | | | (n = 3) |
| | +1 × 10⁷ | 32.48 | 32.90 | 100% |
| | CHO | | | (n = 3) |
| | +1 × 10⁷ | 33.55 | 33.17 | 100% |
| | Vero | | | (n = 3) |

### Example 6: Verification of the Specificity of the Internal Control Probe, Mycobacterial qPCR Detection and Whether there is Cross-Reactivity Between Internal Control Plasmid

### 1. Verification of Specificity of Internal Control Probes

The genome of 11 types of mycobacteria, 8 types of human and animal cells, 2 types of viruses, 9 types of bacteria, and 2 types of mycoplasma were extracted for backup, and the mycobacteria qPCR detection method of this scheme was verified, and the qPCR reaction system and reaction procedure are the same as those in Tables 4 and 5 above.

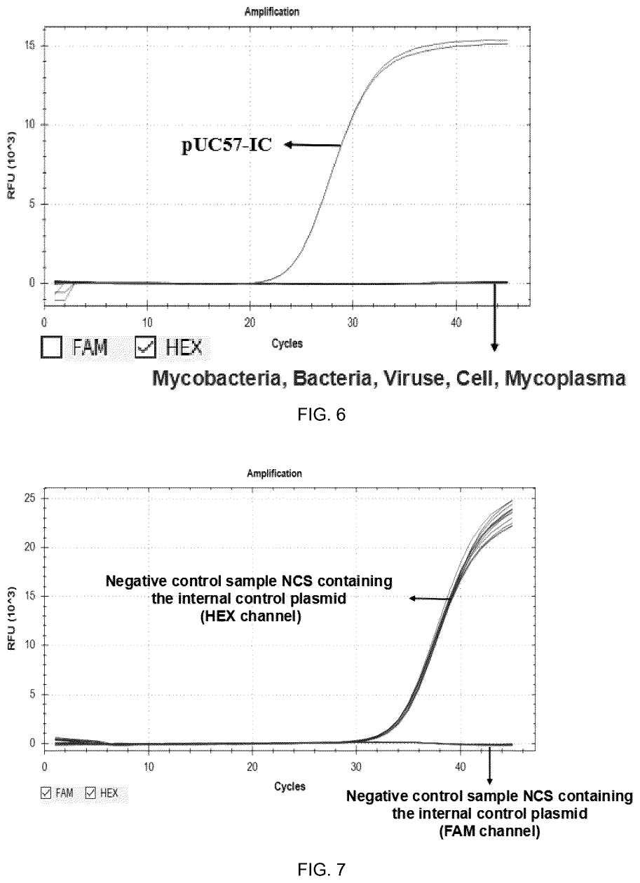

The experimental results are shown in Table 14 and FIG. 6, indicating that the internal control probe did not cross-reactivity with human and animal cells, viruses, mycobacteria, bacteria, and mycoplasma genomes.

TABLE 14
Verification results of cross-reactivity

| Humans and | | | | Common |
|---|---|---|---|---|
| animals cell | virus | Mycobacteria | Other bacteria | mycoplasma |
| PK-15, | HAd-5 | M. avium | Escherichia coli | Mycoplasma |
| MDBK, | AAV-2 | M. terrae | Clostridium | pneumonia |
| Vero, | | M. shimoidei | sporogenes | Mycoplasma |
| CHO-K1, | | M. kansasii | Staphylococcus | Oral |
| 293, 293T, | | M. asiaticum | aureus | |
| BHK-21, | | M. scrofulaceum | Salmonella | |
| MSC | | M. gordonae | typhimurium | |
| | | M. abscessus | Bacillus subtilis | |
| | | M. fortuitum | Pseudomonas | |
| | | M. phlei | aeruginosa | |
| | | M. Bovis BCG | Bacillus cereus | |
| | | | Micrococcus luteus | |
| | | | Corynebacterium | |
| | | | diphtheria | |

Determination of Ct value: NA (no cross-reactivity)

2. Verification of Cross-Reactivity Between Mycobacterial qPCR Detection and Internal Control Plasmid

In the process of extracting the mycobacterial genome, 1 ml of sterile normal saline was taken as a negative control NCS, and an internal control plasmid of 700 copies/sample was introduced, and after the extraction was completed, mycobacterial qPCR was performed to detect, the reaction system and reaction procedures are the same as those in Tables 4 and 5 above.

As shown in FIG. 7, the HEX channel of the NCS of the negative control sample produced a normal amplification curve while the FAM channel did not produce a fluorescence signal, demonstrating that the introduced internal control plasmid did not cross-reactivity with mycobacterial detection, there will be no false positives due to the introduction of internal control plasmid during the measurement.

### Example 7: Application Example of Mycobacterial Detection Method

The samples of the CHO cell seed bank and working bank were detected by qPCR in this protocol to determine whether there is mycobacterial contamination, and the internal control plasmid was chosen to be added during the extraction process, the DNA extraction of detection sample and negative control select the QIAamp® MinElute Virus Spin Kit (QIAGEN 57704), and operate according to the product instructions. The specific method is:
Negative control: 1 ml of sterile saline was taken, and 700 copies of internal control plasmid was added after pretreatment, to extract DNA;
Detection sample: 10⁷ CHO cells from seed banks or working bank sand and 1 ml culture supernatant were taken, and 700 copies of internal control plasmid was added after pretreatment, to extract DNA;
Positive control: positive control plasmids (pUC57—M. tuberculosis, pUC57—M. terrae, pUC57—M. xenopi recombinant plasmid mixture) were taken from the kit diluted to 100 copies/μl, then taken 10 μl to add the qPCR reaction tube, and the internal control plasmid in the kit diluted to 1000 copies/μl, then taken 0.2 μl to add the qPCR reaction tube;
Specifically, pUC57—M. tuberculosis, pUC57—M. terrae, pUC57—M. xenopi recombinant plasmids are transferring the 16S rRNA nucleic acid sequence of Mycobacterium tuberculosis, mycobacterium terrae, mycobacterium xenopi that are capable of monitoring mycobacteria qPCR reaction to the pUC57 plasmid respectively, and obtained after using Plasmid Mini Kit I (OMEGA) to extract. The concentration was determined and the plasmid copy number was calculated, and diluted to 1×10⁸ copies/μl, mix the three in equal volumes and set aside.

No template control: RNase-free water is added to the qPCR reaction tube.

The qPCR reaction system and reaction procedure are the same as those in Tables 4 and 5 above.

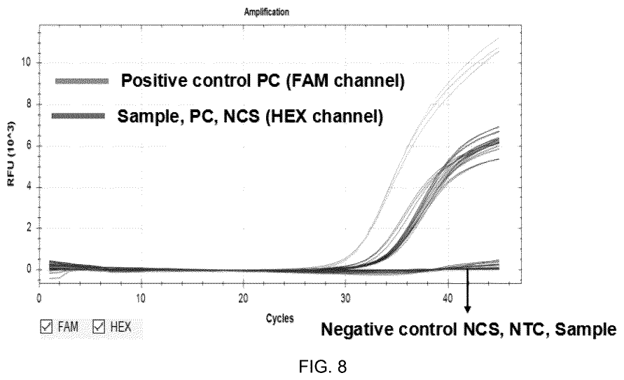

The test results are shown in FIG. 8 and Table 15, according to the quality control result requirements in Table 1 and the sample test result judgment criteria in Table 2, the determination is that there was no mycobacterial contamination in the CHO cell seed bank and the working bank.

TABLE 15
Detection results

| | FAM channel | HEXchannel | |
|---|---|---|---|
| | average Ct | average Ct | The result is |
| Experimental group | value (n = 3) | value (n = 3) | determined |
| Positive control PC | 30.03 | 31.68 | Mycobacteria |
| Negative control NCS | NA | 33.19 | negative |
| No template control NTC | NA | NA | |
| CHO cell seed bank | NA | 32.96 | |
| CHO cell working bank | NA | 33.29 | |

The above specific embodiment describes the implementation of the present disclosure in detail, however, the present disclosure is not limited to the specific details in the above implementation method. Within the scope of the claims and technical conception of the present disclosure, a variety of simple modifications and changes may be made to the technical solution of the present disclosure, and these simple variants fall within the scope of protection of the present disclosure.

What is claimed is:

1. The primer-probe combination for broad-spectrum detection of mycobacteria, wherein including forward primer, reverse primer and probe, the nucleotide sequence of the forward primer is shown in SEQ ID NO. 1 and SEQ ID NO. 2, the nucleotide sequence of the reverse primer is shown in SEQ ID NO. 3, the nucleotide sequence of the detection probe is shown in SEQ ID NO. 4, SEQ ID NO. 5 and SEQ ID NO. 6; the 5′ end of the detection probe is coupled with a first fluorescence group, and the 3′ end is coupled with a quenching group.

2. The primer-probe combination for broad-spectrum detection of mycobacteria according to claim 1, wherein the first fluorescence group is FAM, TET, NED, ROX, CY3, CY5, VIC, JOE, or HEX fluorescence group; the quenching group is TAMRA, NFQ, ECLIPSE, DABCYL, BHQ1, or BHQ2 quenching group.

3. The primer-probe combination for broad-spectrum detection of mycobacteria according to claim 1, wherein the first fluorescence group is FAM fluorescence group, the quenching group is BHQ1 quenching group.

4. The use of the primer-probe combination of claim 1, wherein preparing products for broad-spectrum detection of mycobacteria.

5. A kit for broad-spectrum detection of mycobacteria, wherein including the primer-probe combination of claim 1.

6. The kit for broad-spectrum detection of mycobacteria according to claim 5, wherein also includes an internal control probe and an internal control plasmid, the nucleotide sequence of the internal control probe is shown in SEQ ID NO. 7, the nucleotide sequence of the target fragment in the internal control plasmid is shown in SEQ ID NO. 8; the 5′ end of the internal control probe is coupled with a second fluorescence group, the 3′ end is coupled with a quenching group, and the type of the second fluorescence group is different from the first fluorescence group.

7. A method for broad-spectrum detection of mycobacteria that is not intended for disease diagnosis and treatment, wherein using the kit of claim 5 to detect.

8. The method according to claim 7, wherein including the steps of extracting the nucleic acid in the test product, specifically:
choosing whether to perform a pretreatment process according to the type of sample to be tested; If there is no need for sample pretreatment, it is directly prepared as a test solution for nucleic acid extraction; If sample pretreatment is required, the pretreatment process is to add cell lysate treatment, and collect the pellet as a test product after centrifugation;
the internal control plasmid is added to the test product or qPCR reaction solution, and the nucleic acid in the test product is extracted; the positive control plasmid was added to the internal control plasmid and nuclease-free water and diluted to form a positive control;
taking sterile normal saline as a negative control, adding an internal control plasmid to extract the nucleic acid simultaneously with the test product, or adding an internal control plasmid to the qPCR reaction after extracting nucleic acid; no template control is nuclease-free water, or an internal control plasmid prepared with nuclease-free water;
the first fluorescence group detection channel and the second fluorescence group detection channel were created on the fluorescence quantitative PCR instrument, the qPCR reaction was performed on the test product, no template control, negative control, and positive control respectively, read the Ct value of the two channels respectively; and the determination of the quality control results of positive control, negative control and no template control for the Ct value and the determination of the test results of the test product are performed;
when the first fluorescence group detection channel of the test product is Ct<40 and has a normal amplification curve, the test result of mycobacteria is determined to be positive;
when the first fluorescence group detection channel of the test product has no Ct value and no normal amplification curve, if the internal control plasmid is added in the nucleic acid extraction process, the second fluorescence group detection channel has a normal amplification curve and the difference between the Ct value of the second fluorescence group detection channel in the negative control is not more than 3 cycles; if the internal control plasmid is added to the qPCR reaction, the second fluorescence group detection channel has a normal amplification curve and the difference between the Ct value of the second fluorescence group detection channel in the negative control is not more than 2 cycles, the mycobacterial detection is finally determined negative;
when the first fluorescence group detection channel and the second fluorescence group detection channel of the test product have no Ct value and no normal amplification curve; or when the first fluorescence group detection channel is Ct≥40 and has a normal amplification curve, the second fluorescence group detection channel has no Ct value and no normal amplification curve, the qPCR reaction is determined to be inhibited;
If the first fluorescence group detection channel of the test product is Ct≥40 and has a normal amplification curve, the second fluorescence group detection channel is Ct<40 and has a normal amplification curve, the judgment result is invalid.

9. The method according to claim 8, wherein the criteria for determining the quality control results of positive control, negative control and no template control are:
the amplification status is checked in the corresponding channel, and the threshold line of the amplification curve of the test product is 10% of the Ct value of the target channel in the positive control, and the threshold line of the amplification curve of the internal control plasmid is 10% of the Ct value of the internal control channel in the no template control or negative control;
quality control results requires the first fluorescence group detection channel and the second fluorescence group detection channel of the positive control are both positive; the first fluorescence group detection channel of the negative control is negative, and the second fluorescence group detection channel is positive; the first fluorescence group detection channel of the no template control is negative, the second fluorescence group detection channel is positive when the internal control plasmid is added, and the second fluorescence group detection channel is negative when the internal control plasmid is not added.
